(19) United States
(12) Patent Application Publication
(10) Pub. No.: US 2021/0100530 A1
(43) Pub. Date: Apr. 8, 2021

(54) METHODS AND SYSTEMS FOR DIAGNOSING TENDON DAMAGE VIA ULTRASOUND IMAGING

(71) Applicant: GE Precision Healthcare LLC, Milwaukee, WI (US)

(72) Inventor: Jae Young Park, Yongin-si (KR)

(21) Appl. No.: 16/593,804

(22) Filed: Oct. 4, 2019

(51) Int. Cl. A61B 8/08; G06N 3/02; A61B 5/00; G16H 50/20

(52) U.S. Cl. CPC A61B 8/5207; A61B 8/483; G06N 3/02; A61B 8/467; G16H 50/20; A61B 5/4576; A61B 5/4523

(57) ABSTRACT

Various methods and systems are provided for diagnosing tendon damage using an ultrasound imager. In one example, a method may include acquiring an ultrasound image of an anatomical feature, pairing, via a trained neural network, the acquired ultrasound image to a sample image of a sample anatomical feature, determining a degree of damage of the anatomical feature based on the sample image, and displaying the acquired ultrasound image and the sample image simultaneously.

### FIELD

Embodiments of the subject matter disclosed herein relate to medical imaging, such as ultrasound imaging, and more particularly to identifying tendons and diagnosing damage thereof via ultrasound imaging.

### BACKGROUND

Medical imaging systems are often used to monitor, image, and diagnose a subject. In some examples, the medical imaging system may be a magnetic resonance imaging (MM) system used to diagnose partial or complete tendon rupture in a shoulder of the subject. The diagnosis may include recommending a surgical procedure to repair the rupture. However, MRI systems may be prohibitively costly and may require significant medical expertise to operate and provide diagnoses therefrom.

Ultrasound systems may present a less expensive alternative, but difficulties remain in obtaining accurate scanning of such tendon ruptures. As an example, optimal imaging of the tendon may be obtained only when an angle of incidence of an ultrasound probe is at, or near, 90°. Further, similar to MRI systems, ultrasound systems also require significant medical expertise on the part of the operator.

### BRIEF DESCRIPTION

In one embodiment, a method may include acquiring an ultrasound image of an anatomical feature, pairing, via a trained neural network, the acquired ultrasound image to a sample image of a sample anatomical feature, determining a degree of damage of the anatomical feature based on the sample image, and displaying the acquired ultrasound image and the sample image simultaneously.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example ultrasound imaging system according to an exemplary embodiment;

FIG. 2 shows a flow chart of a method for identifying and diagnosing a tendon in an ultrasound image, according to an embodiment;

FIG. 3 shows a flow chart of a method for using feedback from a gyro sensor to generate an ultrasound image, according to an embodiment;

FIG. 4 shows a flow chart of a method for training a neural network to identify tendons in ultrasound images, according to an embodiment;

FIG. 5 shows a schematic diagram illustrating geometric considerations of adjusting a steering angle based on feedback from the gyro sensor, according to an embodiment;

FIGS. 6A and 6B show example user interface displays for adjusting the steering angle based on feedback from the gyro sensor, according to an embodiment;

FIG. 7 shows a schematic diagram illustrating an example neural network used for identifying a tendon in an ultrasound image, according to an embodiment;

FIG. 8 shows exemplary ultrasound images respectively depicting tendons having varying degrees of damage, according to an embodiment; and

FIG. 9 shows an example user interface display of the display device of the ultrasound imaging system, according to an embodiment.

### DETAILED DESCRIPTION

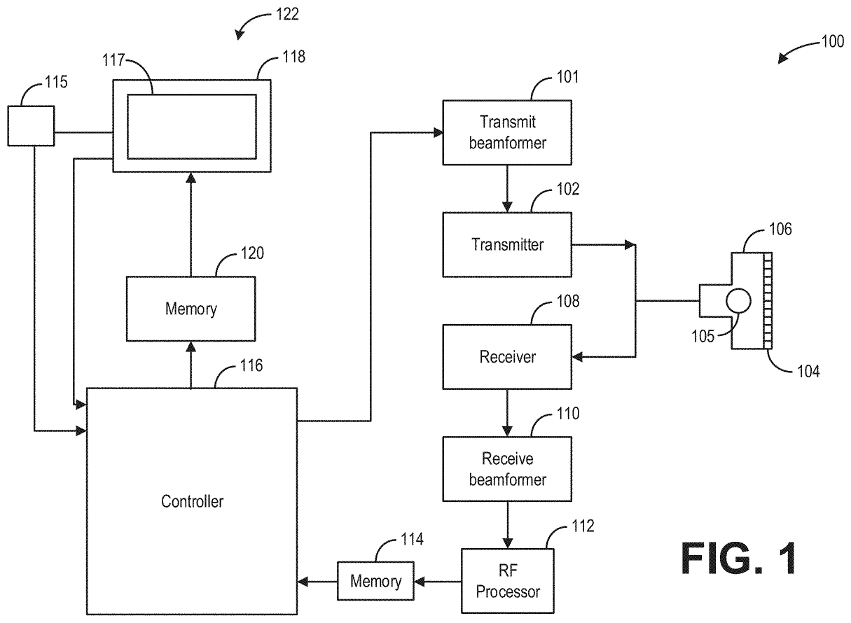

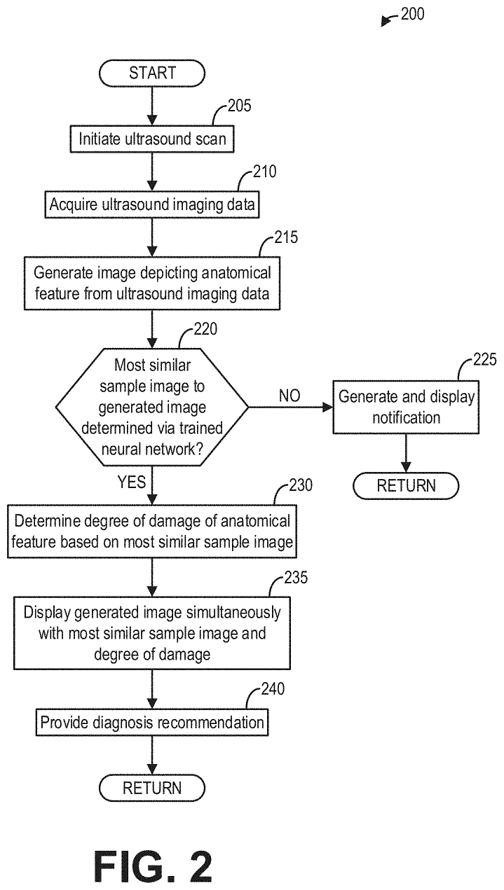

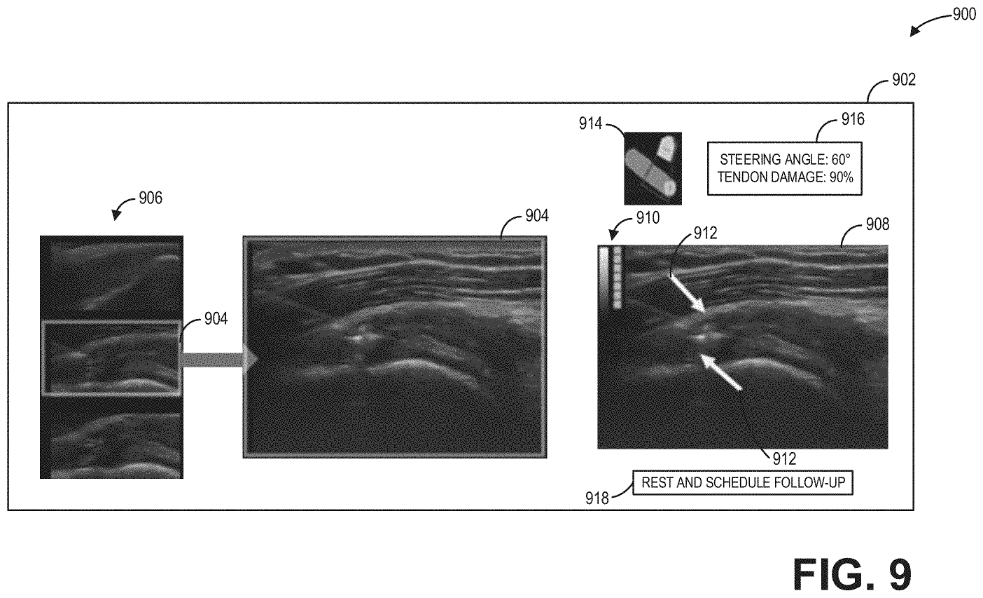

The following description relates to various embodiments of identifying and diagnosing a tendon of a subject via ultrasound imaging. One example ultrasound imaging system for generating imaging data therefor is depicted in FIG. 1. FIG. 2 depicts a method for using the ultrasound imaging system to identify and diagnose the tendon, and then displaying an ultrasound image depicting the tendon. Correspondingly, FIG. 9 depicts an example user interface display of a display device of the ultrasound imaging system, where the generated ultrasound image depicting the tendon may be displayed.

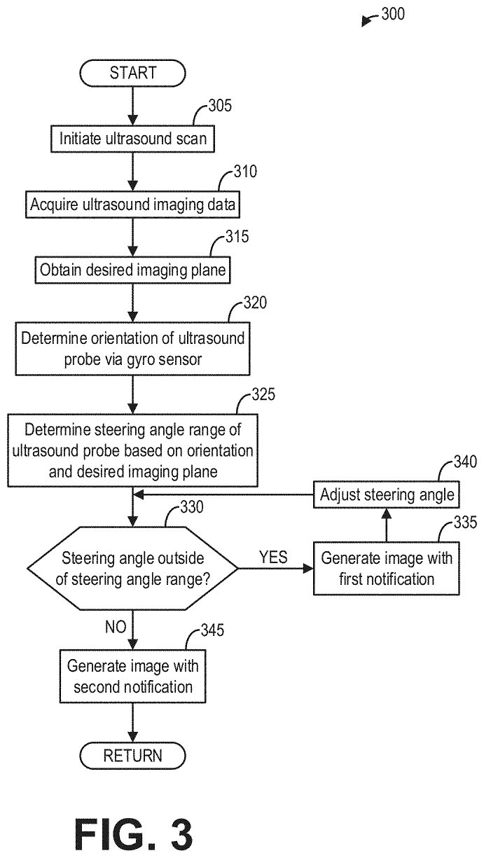

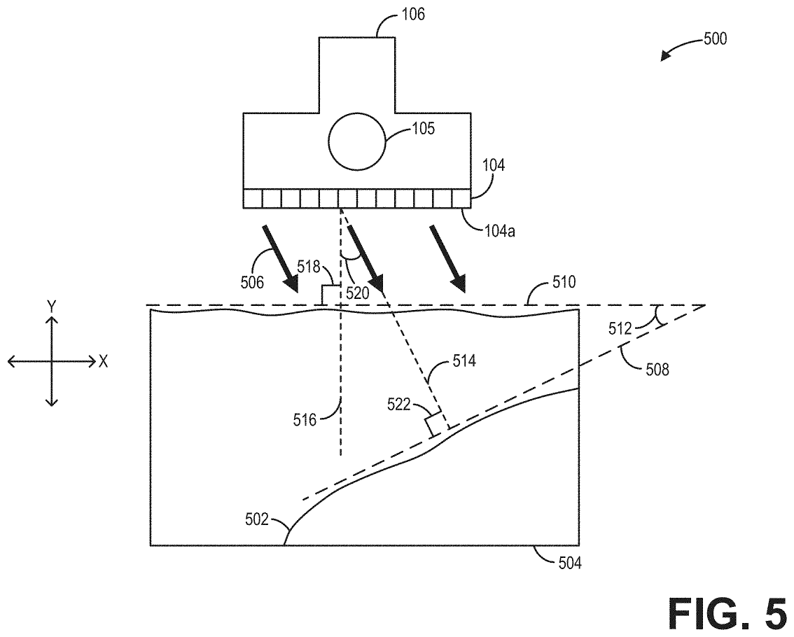

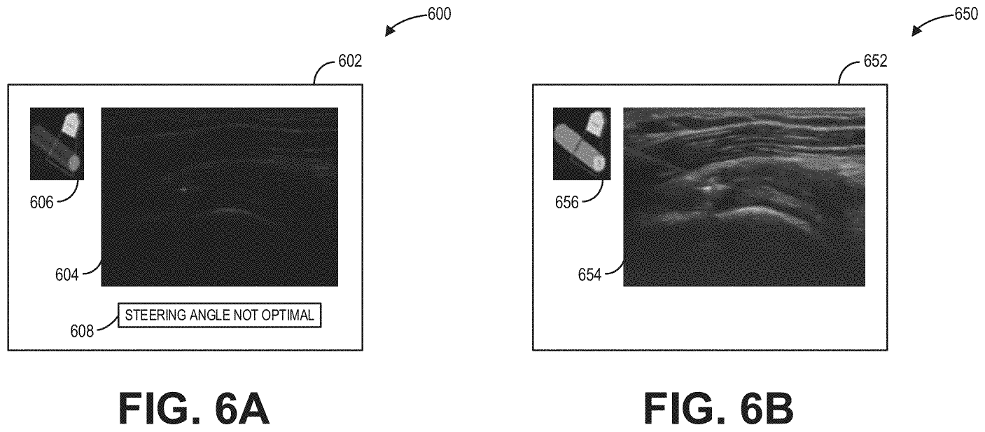

FIG. 3 depicts a method for using an ultrasound probe of the ultrasound imaging system to generate the ultrasound image, where the ultrasound probe may include a gyro sensor. Geometric considerations of an adjustment of a steering angle of the ultrasound probe based on feedback from the gyro sensor are schematically illustrated at FIG. 5. FIGS. 6A and 6B depict example user interface displays for facilitating the adjustment of the steering angle.

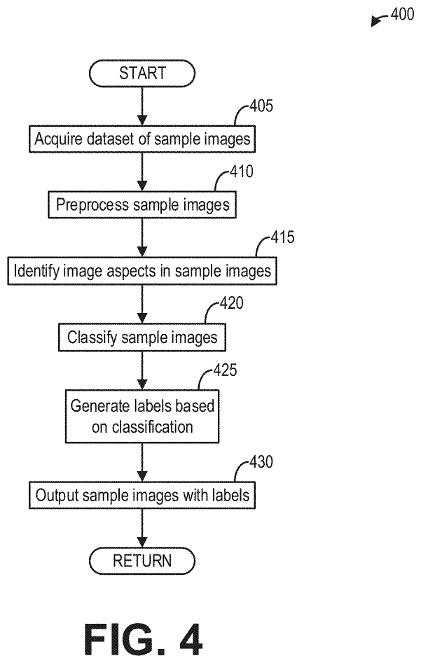

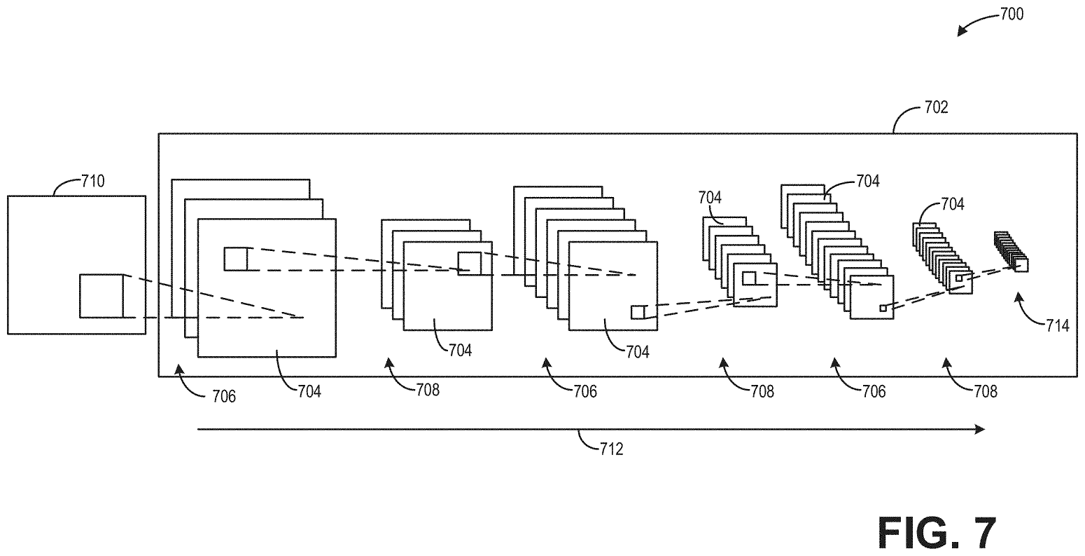

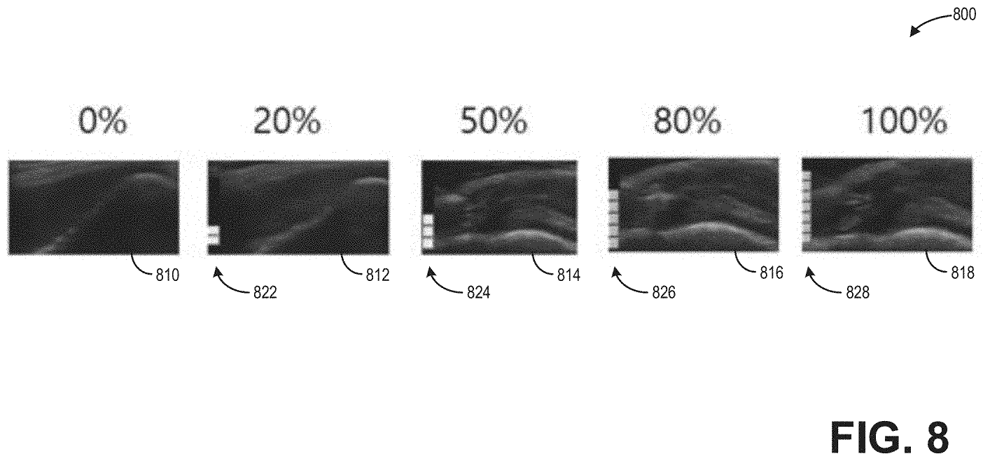

FIG. 4 depicts a method for training an example neural network, such as the example CNN of FIG. 7. The example CNN may receive the ultrasound image and may output a degree of damage of the tendon identified therein. Imaged tendons having various degrees of damage are depicted in FIG. 8.

FIG. 1 depicts a block diagram of a system 100 according to one embodiment. In the illustrated embodiment, the system 100 is an imaging system and, more specifically, an ultrasound imaging system. However, it is understood that embodiments set forth herein may be implemented using other types of medical imaging modalities (e.g., MR, CT, PET/CT, SPECT etc.). Furthermore, it is understood that other embodiments do not actively acquire medical images. Instead, embodiments may retrieve image or ultrasound data that was previously acquired by an imaging system and analyze the image data as set forth herein. As shown, the system 100 includes multiple components. The components may be coupled to one another to form a single structure, may be separate but located within a common room, or may be remotely located with respect to one another. For example, one or more of the modules described herein may operate in a data server that has a distinct and remote location with respect to other components of the system 100, such as a probe and user interface. Optionally, in the case of ultrasound systems, the system 100 may be a unitary system that is capable of being moved (e.g., portably) from room to room. For example, the system 100 may include wheels or be transported on a cart.

In the illustrated embodiment, the system 100 includes a transmit beamformer 101 and transmitter 102 that drives an array of elements 104, for example, piezoelectric crystals, within a diagnostic ultrasound probe 106 (or transducer) to emit ultrasonic signals (e.g., continuous or pulsed) into a body or volume (not shown) of a subject. The elements 104 and the probe 106 may have a variety of geometries. The ultrasonic signals are back-scattered from structures in a body, for example, a tendon in a shoulder, to produce echoes that return to the elements 104. The echoes are received by a receiver 108. The received echoes are provided to a receive beamformer 110 that performs beamforming and outputs a radio frequency (RF) signal. The RF signal is then provided to an RF processor 112 that processes the RF signal. Alternatively, the RF processor 112 may include a complex demodulator (not shown) that demodulates the RF signal to form I/Q data pairs representative of the echo signals. The RF or I/Q signal data may then be provided directly to a memory 114 for storage (for example, temporary storage). The system 100 also includes a system controller 116 that may be part of a single processing unit (e.g., processor) or distributed across multiple processing units. The system controller 116 is configured to control operation of the system 100.

The probe 106 may further comprise a gyro sensor or gyroscope 105 to sense an orientation of the probe 106. Though not explicitly shown, the gyro sensor 105 may be communicably coupled to the system controller 116 wherefrom one or more operating conditions of the gyro sensor 105 may be set, adjusted, and controlled. Further, the system controller 116 may be operable to determine the orientation of the probe 106 relative to a predefined or provided reference plane in real-time based on received signals from the gyro sensor 105.

For example, the system controller 116 may include an image-processing module that receives image data (e.g., ultrasound signals in the form of RF signal data or I/Q data pairs) and processes image data. For example, the image-processing module may process the ultrasound signals to generate two-dimensional (2D) slices or frames of ultrasound information (e.g., ultrasound images) or ultrasound waveforms (e.g., continuous or pulse wave Doppler spectrum or waveforms) for displaying to the operator. When the system 100 is an ultrasound system, the image-processing module may be configured to perform one or more processing operations according to a plurality of selectable ultrasound modalities on the acquired ultrasound information. By way of example only, the ultrasound modalities may include color-flow, acoustic radiation force imaging (ARFI), B-mode, A-mode, M-mode, spectral Doppler, acoustic streaming, tissue Doppler module, C-scan, and elastography. Further, in some examples, the one or more processing operations may include one or more image transforms, such as a Radon transform for identifying linear features in the ultrasound images.

Acquired ultrasound information may be processed in real-time during an imaging session (or scanning session) as the echo signals are received. Additionally or alternatively, the ultrasound information may be stored temporarily in the memory 114 during an imaging session and processed in less than real-time in a live or off-line operation. An image memory 120 is included for storing processed slices or waveforms of acquired ultrasound information that are not scheduled to be displayed immediately. For example, the image memory 120 may store a plurality of sample processed slices or waveforms used for various purposes, e.g., training one or more neural networks, comparing further processed slices or waveforms, etc. The image memory 120 may comprise any known data storage medium, for example, a permanent storage medium, removable storage medium, and the like. Additionally, the image memory 120 may be a non-transitory storage medium.

In operation, an ultrasound system may acquire data, for example, 2D data sets, spectral Doppler data sets, and/or volumetric data sets by various techniques (for example, three-dimensional (3D) scanning, real-time 3D imaging, volume scanning, 2D scanning with probes having positioning sensors, freehand scanning using a voxel correlation technique, scanning using 2D or matrix array probes, and the like). Ultrasound spectrum (e.g., waveforms) and/or images may be generated from the acquired data (at the controller 116) and displayed to the operator or user on the display device 118.

The system controller 116 is operably connected to a user interface 122 that enables an operator to control at least some of the operations of the system 100. The user interface 122 may include hardware, firmware, software, or a combination thereof that enables an individual (e.g., an operator) to directly or indirectly control operation of the system 100 and the various components thereof. As shown, the user interface 122 includes a display device 118 having a display area 117. In some embodiments, the user interface 122 may also include one or more user interface input devices 115, such as a physical keyboard, mouse, and/or touchpad. In one embodiment, a touchpad may be configured to the system controller 116 and display area 117, such that when a user moves a finger/glove/stylus across the face of the touchpad, a cursor atop the ultrasound image or Doppler spectrum on the display device 118 moves in a corresponding manner.

In an exemplary embodiment, the display device 118 is a touch-sensitive display (e.g., touchscreen) that can detect a presence of a touch from the operator on the display area 117 and can also identify a location of the touch in the display area 117. The touch may be applied by, for example, at least one of an individual's hand, glove, stylus, or the like. As such, the touch-sensitive display may also be characterized as an input device that is configured to receive inputs from the operator. The display device 118 also communicates information from the controller 116 to the operator by displaying the information to the operator. The display device 118 and/or the user interface 122 may also communicate audibly. The display device 118 is configured to present information to the operator during or after the imaging or data acquiring session. The information presented may include ultrasound images (e.g., one or more 2D frames), graphical elements, measurement graphics of the displayed images, user-selectable elements, user settings, and other information (e.g., administrative information, personal information of the patient, and the like).

In addition to the image-processing module, the system controller 116 may also include one or more of a graphics module, an initialization module, a tracking module, and an analysis module. The image-processing module, the graphics module, the initialization module, the tracking module, and/or the analysis module may coordinate with one another to present information to the operator during and/or after the imaging session. For example, the image-processing module may be configured to display an acquired image on the display device 118, and the graphics module may be configured to display designated graphics along with the displayed image, such as selectable icons (e.g., image rotation icons) and measurement parameters (e.g., data) relating to the image. The controller may include algorithms and one or more neural networks (e.g., a system of neural networks) stored within a memory of the controller for automatically identifying and diagnosing one or more anatomical features depicted by a generated ultrasound image, as described further below with reference to FIGS. 2-4. In some examples, the controller may include a deep learning module which includes the one or more deep neural networks and instructions for performing the deep learning and feature recognition discussed herein.

The screen of a display area 117 of the display device 118 is made up of a series of pixels which display the data acquired with the probe 106. The acquired data includes one or more imaging parameters calculated for each pixel, or group of pixels (for example, a group of pixels assigned the same parameter value), of the display, where the one or more calculated image parameters includes one or more of an intensity, velocity (e.g., blood flow velocity), color flow velocity, texture, graininess, contractility, deformation, and rate of deformation value. The series of pixels then make up the displayed image and/or Doppler spectrum generated from the acquired ultrasound data.

The system 100 may be a medical ultrasound system used to acquire imaging data of a scanned object (e.g., a tendon). The acquired image data may be used to generate one or more ultrasound images which may then be displayed via the display device 118 of the user interface 122. The one or more generated ultrasound images may include one or more 2D frames, for example. Specifically, the image-processing module discussed above may be programmed to generate and display the one or more 2D frames.

In general, ultrasound imaging systems requires significant expertise on the part of an operator to orient and use an ultrasound probe (e.g., 106) to optimally image, identify, and diagnose an anatomical feature. For example, for optimal imaging, it may be desirable for the ultrasound beam to meet the anatomical feature to be imaged at a 90° angle. Said another way, it may desirable to adjust a steering angle of the ultrasound probe such that an angle of incidence is 90°. However, achieving a desired angle of incidence may be difficult via user control of such a non-intuitive parameter (e.g., the steering angle). In some examples, such inherent difficulties may result in suboptimal visualization of the anatomical feature. Further, even assuming optimal imaging, certain anatomical features may be difficult to identify and diagnose, and differing operators (e.g., medical professionals) with varying levels of expertise may provide conflicting diagnoses for a given anatomical feature, leading to inconsistent treatment. For example, the anatomical feature may be a ruptured tendon. Upon imaging the ruptured tendon, one medical professional may determine 75% damage to the tendon (e.g., a partial rupture) and another medical professional may determine 100% damage to the tendon (e.g., a complete rupture).

According to embodiments disclosed herein, the above-described issues may be at least partly addressed by methods and systems for assisting a user in obtaining ultrasound imaging data via an ultrasound probe and, based on the ultrasound imaging data, using a trained neural network to automatically identify and diagnose an anatomical feature (e.g., a tendon). Feedback from a gyro sensor included in the ultrasound probe may be used to determine a steering angle of the ultrasound probe, which may then be presented to the user. The user may then be enabled to adjust the steering angle and optimize an image generated from the obtained ultrasound imaging data in real-time. The trained neural network may then pair the generated image with a sample image depicting a similar anatomical feature (e.g., a similarly damaged tendon), whereby each of the generated image and the sample image may be simultaneously presented to the user. In some examples, based on the sample image, the trained neural network may further determine a degree of damage of the anatomical feature depicted by the generated image and a diagnosis recommendation may be provided therefor. In this way, a cost of medical imaging may be reduced via an ultrasound imaging system which may be operated with less expertise and training, and which may be cheaper than a conventional magnetic resonance imaging system. Further, by employing a trained neural network, a diagnosis may be obtained for a ruptured tendon (or other damaged anatomical feature) in a more consistent and predictable manner.

Referring now to FIGS. 2-4, methods are depicted to identify and diagnose imaged anatomical features via a trained neural network. It will be appreciated by those skilled in the art that, though FIGS. 2-4 are described with reference to ultrasound imaging, one or more of the features described therein may be applicable to other medical imaging technologies and that the description therein should not be considered as limited to ultrasound imaging systems.

Referring now to FIG. 2, a method 200 is depicted for identifying and diagnosing an anatomical feature, e.g., a tendon in a shoulder of a subject, depicted by an ultrasound image generated from ultrasound imaging data acquired from an ultrasound imaging system. Thereafter, the generated ultrasound image may be displayed to a user at a display device.

Method 200 is described below with regard to the systems and components depicted in FIG. 1, though it should be appreciated that method 200 may be implemented with other systems and components without departing from the scope of the present disclosure. In some embodiments, method 200 may be implemented as executable instructions in any appropriate combination of the imaging system 100, an edge device (e.g., an external computing device) connected to the imaging system 100, a cloud in communication with the imaging system, and so on. As one example, method 200 may be implemented in non-transitory memory of a computing device, such as the controller (e.g., processor) of the imaging system 100 in FIG. 1.

Method 200 may begin at 205 where an ultrasound scan may be initiated. The ultrasound scan may include the ultrasound beam being emitted from transducer elements (e.g., 104) of an ultrasound probe (e.g., 106). The ultrasound beam may be directed toward a body of a subject, such as a patient. Therein, the ultrasound beam may be echoed from an anatomical feature, such as a tendon in a shoulder of the patient. It will be appreciated by those skilled in the art that any tendon or other anatomical feature in the body may be accordingly imaged in this way, and that method 200 should not be understood as being limited to a tendon in a shoulder.

At 210, method 200 may include acquiring ultrasound imaging data from a probe of an ultrasound imager. The ultrasound imager may be one or more components of the imaging system 100 shown in FIG. 1, for example. In such examples, the probe may be the ultrasound probe 106. The probe may be used to image an anatomical feature, for example, a tendon in a shoulder, by collecting an ultrasound beam echoed therefrom. Specifically, the acquired ultrasound imaging data may include ultrasound echoes of ultrasound waves transmitted by transducer elements (e.g., elements 104 of FIG. 1) of the probe of the ultrasound imager. In some examples, the imaging data may include volumetric ultrasound data. The ultrasound imaging data may be received by the controller (e.g., 116) communicably coupled to the ultrasound probe. Further, the ultrasound imaging data may be based on one or more positional parameters of the ultrasound probe, such as a distance of the ultrasound probe from the tendon and an orientation of the ultrasound probe relative to the shoulder. As such, in some examples, the ultrasound probe may further include a gyro sensor (e.g., 105) configured to send feedback to the controller. As described below with reference to FIG. 3, the feedback may be used by the system controller to determine the orientation of the ultrasound probe relative to the shoulder. In this way, a steering angle of the ultrasound probe may be manually adjusted by the user based on the feedback from the gyro sensor (e.g., the user may use the determined orientation in real-time to adjust the steering angle to a value optimal for imaging the anatomical feature). In other examples, the steering angle of the ultrasound probe may be automatically adjusted by the controller to a value optimal for imaging of the anatomical feature based on the feedback from the gyro sensor.

At 215, method 200 may include generating an ultrasound image depicting the anatomical feature (e.g., tendon) from the ultrasound imaging data. In some examples, the generated ultrasound image may be a 2D image slice of a volume (e.g., from the volumetric ultrasound data) corresponding to a targeted slice of the volume (e.g., a sagittal, frontal, or transverse plane of a shoulder of the patient). As discussed below with reference to FIG. 3, the ultrasound image may be generated from ultrasound imaging data optimized for quality and clarity, for example, by manually or automatically adjusting the steering angle of the ultrasound beam such that the angle of incidence is at, or near, 90°.

At 220, method 200 may include determining whether a most similar sample image to the generated image is determined via a trained neural network. The trained neural network may be configured to separate visual characteristics from content of an image. As such, the trained neural network may be a convolutional neural network, such as the convolutional neural network 702 of FIG. 7. The trained neural network may include an object detection algorithm, used for pairing the generated ultrasound image with one of a plurality of sample images. The plurality of sample images may each be a sample ultrasound image slice depicting a sample anatomical feature, such that the plurality of sample images may be compared by the neural network to the generated ultrasound image to determine the most similar sample image and thereby match the most similar sample image to the generated ultrasound image. The generated ultrasound image may thus be an input of the trained neural network. In some examples, the angle of incidence may be an additional input of the trained neural network. For example, the trained neural network may infer a quality of the generated ultrasound image by how far the inputted angle of incidence is from an optimal value, such as 90°, and weight the generated ultrasound image accordingly. The angle of incidence, in turn, may depend upon the feedback from the gyro sensor. In this way, the trained neural network may rely on the feedback from the gyro sensor to determine a level of confidence in the most similar sample image.

The trained neural network may identify one or more image aspects of the anatomical feature depicted by the generated ultrasound image based on the most similar sample image. Specifically, the trained neural network may pair a corresponding one of one or more predetermined image aspects of the sample anatomical feature depicted by the most similar sample image to each of the one or more image aspects of the anatomical feature depicted by the generated ultrasound image. In some examples, the one or more image aspects may include one or more tendon features (e.g., individual fiber bundles, partial ruptures, complete ruptures, etc.). In some examples, each of the plurality of sample images may depict a tendon of a shoulder. As such, one or more of the plurality of sample images may depict a non-damaged tendon (e.g., not having a partial or complete rupture), and each remaining sample image of the plurality of sample images may depict a tendon having a partial or complete rupture. In other examples, one or more of the plurality of sample images may depict one or more anatomical features not including a tendon (e.g., to provide minority classes and prevent biasing of neural network training).

Upon identifying the one or more image aspects of the anatomical feature depicted by the generated ultrasound image, each of the one or more identified image aspects may be labelled on the generated ultrasound image. The labelling may include placement of one or more visual indicators (e.g., arrows, circles, rectangles, high-contrast shading, etc.) respectively corresponding to the one or more identified image aspects. In this way, the trained neural network may identify and localize the anatomical feature within the generated ultrasound image.

In some examples, a given sample image may be considered a most similar sample image when the degree of similarity of the given sample image to the generated sample image is within a matching threshold. In such examples, there may be instances in which a subset of the plurality of sample images match the generated ultrasound image within the image threshold. As such, the most similar sample image to the generated ultrasound image may then be determined as a sample image of the subset having the highest degree of similarity to the generated ultrasound image. In alternative examples, the most similar sample image may be the sample image having the highest degree of similarity to the generated ultrasound image regardless of meeting the matching threshold. In further examples in which none of the plurality of sample images match the generated ultrasound image within the matching threshold (for example, as a result of a poor ultrasound imaging or imaging of an anatomical feature not represented in the plurality of sample images), or for example when no image aspects are identified and labeled via the trained neural network, no most similar sample image to the generated ultrasound image may be determined via the trained neural network. In such examples, method 200 may proceed to 225 to generate and display, at a display device (e.g., 118), a notification indicating no matching sample image was determined. However, in some examples, the generated ultrasound image may be displayed regardless of the trained neural network being unable, as a result of not identifying the most similar sample image, to identify and label one or more image aspects of the depicted anatomical feature. Method 200 may then end.

Returning to 220, if the most similar sample image to the generated ultrasound image is determined via the trained neural network, method 200 may proceed to 230 to determine a degree of damage of the anatomical feature depicted by the generated ultrasound image based on the most similar sample image. The degree of damage may be a percentage value, corresponding to a relative amount of damage to a given anatomical feature. As an example, the anatomical feature may be a tendon in a shoulder, such that a degree of damage of 0% may indicate a non-damaged tendon, a degree of damage of 50% (or any value greater than 0% and less than 100%) may indicate a tendon having a partial rupture, and a degree of damage of 100% may indicate a tendon having a complete rupture. In some examples, the degree of damage may be measured with any sort of numerical scale. Further, each of the plurality of sample images may respectively depict a sample tendon associated with a sample degree of damage. As such, the sample degrees of damage of the plurality of sample images may range from indicating no tendon damage to indicating complete tendon rupture. In some examples, each of the sample degrees of damage of the plurality of sample images may be determined by one or more medical professionals. In this way, accumulated medical expertise may be relied upon to determine the degree of damage of the anatomical feature depicted by the generated ultrasound image.

Because anatomical features respectively depicted by the plurality of sample images may each be associated with a respective degree of damage, upon determination by the trained neural network of the most similar sample image to the generated ultrasound image, the degrees of damage of each of the most similar sample image and the generated ultrasound image may be assumed to be approximately equal. That is, the degree of damage of the anatomical feature depicted by the generated ultrasound image may be determined to be the same as the degree of damage of the sample anatomical feature of the most similar sample image. In this way, by pairing the generated ultrasound image to the most similar sample image, the trained neural network may ultimately allow determination of the degree of damage of the anatomical feature depicted by the generated ultrasound image.

At 235, method 200 may include displaying, at a display area (e.g., 117) of the display device (e.g., 118), the generated ultrasound image simultaneously with the most similar sample image and an indication of the determined degree of damage of the anatomical feature. In some examples, the indication of the determined degree of damage may be a visual indicator. As an example, a graphical bar may indicate the determined degree of damage, where filling of the bar may be proportional to the determined degree of damage. Thus, in examples wherein the anatomical feature is a tendon in a shoulder, an empty bar may indicate no tendon damage, a partially filled bar may indicate a partial rupture, and a completely filled bar may indicate a complete rupture. In some examples, the indication of the degree of damage may be a color. Again considering the example of a tendon in a shoulder, a green color may indicate a non-damaged tendon, a yellow color may indicate a tendon having a partial rupture, and a red color may indicate a tendon having a complete rupture. In some examples, the degree of damage may be associated with a symbol, such as an emoticon (e.g., a “happy” face indicating no tendon damage, a “neutral” face indicating a partial rupture, a “sad” face indicating a complete rupture, etc.). Additionally or alternatively, the indication of the determined degree of damage may include a numerical value (e.g., a percentage) or a verbal description (e.g., “no damage,” “partial rupture,” “complete rupture,” etc.). It will thus be appreciated by those skilled in the art that the degree of damage may be representable in numerous ways (e.g., visually, numerically, verbally, etc.) without departing from the scope of the present disclosure. In this way, a user of the system (e.g., 100) may be automatically presented with the generated ultrasound image aligned to the most similar sample image for comparison. Additionally, the indication of the determined degree of damage may further assist a medical professional (e.g., the operator of the system 100) in diagnosing the anatomical feature.

In some examples, additional information and/or user-actuatable features may be provided at the display area (e.g., 117) of the display device (e.g., 118). For example, an indication of a quality of the generated ultrasound image or of a degree of matching to the most similar sample image may be displayed. As another example, the steering angle of the ultrasound beam and/or a visual or verbal indication of the steering angle being outside of an optimal steering angle range (e.g., based on a determined angle of incidence) may be displayed. As yet another example, in cases wherein the subset of the plurality of sample images is determined to be within the matching threshold, remaining sample images in the subset may be provided in addition to the most similar sample image (e.g., as a grid display, selectable from a dropdown or scrollable menu, etc.).

As yet another example, at 240, method 200 may include providing a diagnosis recommendation for the anatomical feature based on the determined degree of damage for display at the display area (e.g., 117) of the display device (e.g., 118). For example, the diagnosis recommendation may include diagnosing a tendon in a shoulder of a patient based on the determined degree of damage. In some examples, one or more diagnosis thresholds may be set to provide ranges for different diagnoses corresponding to different degrees of damage. As a first example, a diagnosis recommendation for the degree of damage of 0% (e.g., lower than a first diagnosis threshold) may be a “wait and see” approach, wherein the patient monitors a shoulder pain and returns to a provider at a later date. As a second example, a diagnosis recommendation for the degree of damage of 50% (e.g., between the first diagnosis threshold and a second diagnosis threshold) may be to avoid excess stress on the shoulder having the damaged tendon and seeking additional rest. As a third example, a diagnosis recommendation for the degree of damage of 100% (e.g., higher than the second diagnosis threshold) may be a surgical procedure to repair the damaged tendon. Method 200 may then end.

In this way, a method for an ultrasound imaging system is provided by an embodiment of the present disclosure for acquiring an ultrasound image depicting an anatomical feature (e.g., a tendon in a shoulder) and then identifying one or more image aspects (e.g., a rupture) of the anatomical feature by pairing, via a trained neural network, the ultrasound image to a sample ultrasound image of a sample anatomical feature. A degree of damage of the anatomical feature may then be determined based on the sample image, and the ultrasound image, the sample image, and the degree of damage may be displayed simultaneously at a display device to facilitate comparison by a user of the ultrasound imaging system.

Referring now to FIG. 3, a method 300 is depicted for using feedback from a gyro sensor included in an ultrasound probe to facilitate generation of an ultrasound image. In some examples, the generated ultrasound image may then be passed to a trained neutral network for identification and diagnosis of an anatomical feature depicted therein (e.g., following method 300). As such, in some examples, method 300 may be used in place of 200 to 215 of method 200, whereby method 200 may continue at 220 following completion of method 300.

Method 300 is described below with regard to the systems and components depicted in FIG. 1, though it should be appreciated that method 300 may be implemented with other systems and components without departing from the scope of the present disclosure. In some embodiments, method 300 may be implemented as executable instructions in any appropriate combination of the imaging system 100, an edge device (e.g., an external computing device) connected to the imaging system 100, a cloud in communication with the imaging system, and so on. As one example, method 300 may be implemented in non-transitory memory of a computing device, such as the controller (e.g., processor) of the imaging system 100 in FIG. 1.

Method 300 may begin at 305 where an ultrasound scan may be initiated. The ultrasound scan may include the ultrasound beam being emitted from transducer elements (e.g., 104) of an ultrasound probe (e.g., 106). The ultrasound beam may be directed toward a body of a subject, such as a patient. Therein, the ultrasound beam may be echoed from an anatomical feature, such as a tendon in a shoulder of the patient. It will be appreciated by those skilled in the art that any tendon or other anatomical feature in the body may be accordingly imaged in this way, and that method 300 should not be understood as being limited to a tendon in a shoulder.

At 310, method 300 may include acquiring ultrasound imaging data from a probe of an ultrasound imager. The ultrasound imager may be one or more components of the imaging system 100 shown in FIG. 1, for example. In such examples, the probe may be the ultrasound probe 106. The probe may be used to image an anatomical feature, for example, a tendon in a shoulder, by collecting an ultrasound beam echoed therefrom. Specifically, the acquired ultrasound imaging data may include ultrasound echoes of ultrasound waves transmitted by transducer elements (e.g., elements 104 of FIG. 1) of the probe of the ultrasound imager. In some examples, the imaging data may include volumetric ultrasound data. Specifically, the ultrasound imaging data may be received by the controller (e.g., 116) communicably coupled to the ultrasound probe.

At 315, method 300 may include obtaining a desired imaging plane. In some examples, the desired imaging plane may be selected by an operator of the system (e.g., 100) based on previous experience and a desired imaging area. For example, if a shoulder of a patient is the desired imaging area, the operator may hold the ultrasound probe parallel to a surface of the shoulder and indicate that the ultrasound probe is positioned perpendicular to the surface of the shoulder. The controller (e.g., 116) may then determine the desired imaging place based on stored anatomical information. For example, if the tendon of the shoulder roughly corresponds to the desired imaging plane, the controller may determine the desired imaging plane based on the surface of the shoulder provided thereto by the operator. Additionally or alternatively, the operator may provide the system with the anatomical feature to be imaged (e.g., a tendon of a shoulder) and a position of the patient (e.g., sitting up, lying down, etc.) from which the controller may determine the desired imaging plane.

At 320, method 300 may include determining an orientation of the ultrasound probe (e.g., 106) via the gyro sensor (e.g., 105). The gyro sensor may be configured to send feedback to the controller, wherefrom the controller may determine one or more positional parameters of the ultrasound probe, such as a distance of the ultrasound probe from the tendon and the orientation of the ultrasound probe relative to the desired imaging plane. The ultrasound imaging data may be based on the one or more positional parameters of the ultrasound probe, such that a given orientation of the ultrasound probe may provide higher quality imaging over another orientation of the ultrasound probe.

At 325, method 300 may include determining a steering angle range of the ultrasound probe (e.g., 106) based on the orientation and the desired imaging plane. In general, for optimal ultrasound imaging, an angle of incidence (that is, an angle between the ultrasound beam and an object being imaged) should be at, or near, 90°. However, the angle of incidence is typically adjusted by adjusting a steering angle of the ultrasound beam. Since manually adjusting the steering angle of the ultrasound beam to correspondingly adjust the angle of incidence may be a non-intuitive process, especially to operators with little experience, and since the angle of incidence may be determined based on the orientation and the desired imaging plane, it may be desirable to automatically determine the optimal steering angle needed for the angle of incidence to be 90° based on the orientation and the desired imaging plane (further geometrical considerations of determining the optimal steering angle are described below with reference to FIG. 5). The steering angle range may then be determined based on the optimal steering angle as a range of steering angles in which optimal ultrasound imaging of the anatomical feature may be accomplished. For example, the steering angle range may be between 15° less than the optimal steering angle and 15° greater than the optimal steering angle. As another example, the steering angle range may be between 10° less than the optimal steering angle and 10° greater than the optimal steering angle. As yet another example, the steering angle range may be between 5° less than the optimal steering angle and 5° greater than the optimal steering angle.

At 330, method 300 may include determining whether the steering angle is outside the steering angle range. If the steering angle is outside the steering angle range, method 300 may proceed to 335 to generate an ultrasound image with a first notification indicating the steering angle outside the steering angle range. In some examples, the first notification may be a verbal message warning the operator that the steering angle is suboptimal and/or instructing the operator to adjust the steering angle to within the steering angle range. In additional or alternative examples, the first notification is a visual notification, such as a colored symbol (e.g., a color bar). As such, when the steering angle is outside of the steering angle range, the color bar may be set to a first color (e.g., red).

At 340, method 300 may include adjusting the steering angle. In some examples, the steering angle may be manually adjusted by the operator, where the operator may request an adjusted steering angle based on the first notification. In additional or alternative examples, the steering angle may be automatically adjusted by the controller (e.g., 116) based on the orientation and the desired imaging plane. Method 300 may then return to 330.

Returning to 330, if the steering angle is within the steering angle range, method 300 may proceed to 345 to generate the ultrasound image with the second notification indicating the steering angle within the steering angle range. In some examples, the second notification may be a verbal message informing the operator that the steering angle is set to a value for optimal ultrasound imaging and that the operator should maintain a current steering angle. In additional or alternative examples, the second notification is a visual notification, such as the colored symbol (e.g., the color bar). As such, when the steering angle is within the steering angle range, the color bar may be set to a second color (e.g., green). Method 300 may then end.

In this way, a method is provided for manually or automatically adjusting a steering angle of an ultrasound probe of an ultrasound imaging system based on feedback from a gyro sensor included in the ultrasound probe, such that an anatomical feature may be optimally imaged.

Referring now to FIG. 4, a method 400 is depicted for training a neural network to identify an anatomical feature (e.g., a tendon) depicted by an ultrasound image. Specifically, the neural network may be trained to select, from a plurality of sample images, a most similar sample image to the ultrasound image. In some examples, method 400 may be used to train the neural network used at 220 of FIG. 2. In additional or alternative examples, the neural network may be the convolutional neural network 702 of FIG. 7.

Method 400 is described below with regard to the systems and components depicted in FIG. 1, though it should be appreciated that method 400 may be implemented with other systems and components without departing from the scope of the present disclosure. In some embodiments, method 400 may be implemented as executable instructions in any appropriate combination of the imaging system 100, an edge device (e.g., an external computing device) connected to the imaging system 100, a cloud in communication with the imaging system, and so on. As one example, method 400 may be implemented in non-transitory memory of a computing device, such as the controller (e.g., processor) of the imaging system 100 in FIG. 1.

At 405, method 400 may include acquiring a dataset of sample images for training the neural network. Each sample image in the dataset may be a sample ultrasound image slice depicting a sample anatomical feature. In some examples, each sample image in the dataset may depict a particular anatomical feature of interest (e.g., a tendon of a shoulder), such that the dataset of sample images may constitute a suitable training set for diagnosing the anatomical feature of interest. As such, one or more of the sample images in the dataset may depict a non-damaged tendon (e.g., not having a partial or complete rupture), and each remaining sample image in the dataset may depict a tendon having a partial or complete rupture. Further, each of the sample images depicting a tendon may be associated and labeled with a degree of damage of the tendon. In some examples, the degree of damage may be set on a numerical scale ranging from an indication of no tendon damage (for example, 0%) to an indication of complete tendon rupture (for example, 100%). In some examples, each of the degrees of damage of the sample images may be determined by one or more medical professionals. In this way, accumulated medical expertise may be relied upon to train the neural network determine the degree of damage of the anatomical feature of interest. In other examples, one or more sample images in the dataset may depict one or more anatomical features not including a tendon (e.g., to provide minority classes and prevent biasing of neural network training).

At 410, method 400 may include preprocessing the sample images for neural network training. In some examples, the controller (e.g., 116) may be configured to parse the sample images into respective matrices of image patches, from which one or more image aspects may be extracted. In additional or alternative examples, data augmentation may be performed on the sample images. In general, data augmentation routines are employed to add features to a dataset (e.g., in preparation for neural network training) without collecting new data. In object detection applications, simple image processing techniques, such as transformations, rotations, reflections, and color alterations may be applied to images in the dataset to improve identification of desired objects. As such, data augmentation may provide an increased number of sample images without further input from medical professionals, further ultrasound scans, etc. It will be appreciated that numerous data augmentation routines are well-known to those skilled in the art and will therefore be referenced only generally herein as relates to the described embodiments. In alternative examples, rotational invariance and robustness to noise may be incorporated into neural network training, such that no data augmentation is employed.

At 415, method 400 may include identifying, via the neural network, one or more image aspects in the sample images. The one or more image aspects may be any image feature indicating and/or characterizing the anatomical feature of interest. In examples wherein the anatomical feature is a tendon, the one or more image aspects may include one or more tendon features (e.g., individual fiber bundles, partial ruptures, complete ruptures, etc.). Additionally, the neural network may be trained to identify further image aspects, such as image aspects indicating or characterizing other anatomical features, non-anatomical features, or background features. As described below with reference to FIG. 7, a feature extraction module may cycle convolution, non-linear transformation, rectification, and pooling of the sample images (or image patches) to extract the one or more image aspects from the sample images. In this way, the neural network may be trained to identify one or more image aspects corresponding to an anatomical feature depicted by an ultrasound image input.

At 420, method 400 may include classifying the sample images via the neural network. Specifically, after identifying the one or more image aspects for a given sample image, the neural network may be trained to classify each sample image according to a most similar sample image from the remaining sample images. As described below with reference to FIG. 7, a classification module may include a fully-connected layer which receives sample images output from the feature extraction module and classifies each sample image based on probabilities that each other sample image is the most similar sample image thereto (where the probabilities may be determined based on a probability distribution generated by the softmax function). In this way, the neural network may be trained to pair a most similar sample image to each sample image in the dataset.

At 425, method 400 may include generating one or more labels on the sample images based on the neural network classification. Upon classification, the neural network may label the one or more image aspects on the sample images by generating corresponding visual indicators (e.g., arrows, boxes, circles, shading, etc.). In some examples, each of the visual indicators may be associated with a verbal description of the image aspect (e.g., “partial rupture,” “complete rupture,” etc.). Further, once the most similar sample image to a particular sample image is determined by the neural network, the degree of damage of the tendon depicted by the most similar sample image may be obtained. During training, a degree of damage of a tendon depicted by the most similar sample image may be validated against the degree of damage of the tendon depicted by the particular sample image. In practice, the degree of damage of the tendon depicted by the most similar sample image may be assumed to be the degree of damage of the tendon depicted by the particular sample image as well. Further, once the degree of damage is determined in this way, the particular sample image may be associated or labeled with the determined degree of damage. In this way, by pairing the most similar sample image to each sample image in the dataset, the neural network may correspondingly be trained to determine and label a degree of damage of an anatomical feature depicted by a given ultrasound image.

At 430, method 400 may include outputting the sample images with the one or more labels. In some examples, each of the sample images with the one or more labels may be passed to a display device (e.g., 118) for display, along with the most similar sample image thereto for comparison. Method 400 may then end.

In this way, a method is provided for training a neural network to determine a degree of damage of an anatomical feature (e.g., a tendon) depicted by an ultrasound image based on identification of a most similar sample image. The most similar sample image may then be provided to an operator of an ultrasound imaging system for comparison with the ultrasound image.

Referring now to FIG. 5, a schematic diagram 500 depicts geometric considerations of adjusting a steering angle 520 of one or more ultrasound beams 506 emitted by the transducer elements 104 of the ultrasound probe 106. Such geometric considerations may be utilized by the ultrasound imaging system as described with reference to FIG. 1 and the methods described with reference to FIGS. 2 and 3. Specifically, the ultrasound beam(s) 506 may be directed toward an anatomical feature of interest 502 within a body 504 of a subject. The ultrasound beam(s) 506 may reflect off of at least a portion of the anatomical feature of interest 502, such that the at least a portion of the anatomical feature of interest 502 may be imaged by processing echoes of the ultrasound beam(s) 506 received at a surface 104a of the transducer elements 104. Perpendicular X and Y axes are shown at FIG. 5 for indicating relative relationships between the various elements further depicted therein.

An imaging plane 508 may be defined approximately parallel to a surface of the anatomical feature of interest 502. In examples wherein the geometric considerations depicted by FIG. 4 are utilized by the ultrasound imaging system (e.g., 100), the imaging plane 508 may be provided by an operator of the ultrasound imaging system. Alternatively, the imaging plane 508 may be inferred from an operator input. Further, a transducer plane 510 may be defined parallel to each of the X axis and the surface 104a of the transducer elements 104 of the ultrasound probe 106. At an intersection of the imaging plane 508 and the transducer plane 510, an angle 512 may be defined. Further, a line 514 may be defined parallel to the ultrasound beam(s) 506, and a line 516 may be defined parallel to the Y axis and perpendicular to the transducer plane 510 (e.g., such that an angle 518 defined between the transducer plane 510 and the line 516 is 90°). At an intersection of the line 514 and the line 516, a steering angle 520 may be defined. The steering angle 520 may thus be characterized as an angle at which the ultrasound beam(s) 506 are emitted from the transducer elements 104 of the ultrasound probe 106 relative to an orientation of the ultrasound probe 106. Further, at an intersection of the imaging plane 508 and the line 514, an angle of incidence 522 may be defined. The angle of incidence 522 may thus be characterized as an angle between the ultrasound beam(s) 506 and the imaging plane 508 corresponding to the anatomical feature of interest 502.

For optimal imaging of the anatomical feature of interest 502, the angle of incidence 522 should be as close as possible to 90°. Geometrically, to obtain a value of 90°, the angle 512 and the steering angle 520 must be equal. In some examples, the angle 512 may be determined by providing an ultrasound imaging system (e.g., the ultrasound imaging system 100 of FIG. 1) with the imaging plane 508 (or one or more parameters which the ultrasound imaging system 100 may use to determine the imaging plane 508) and then setting a reference orientation 524 of the gyro sensor 105 to be parallel to the Y axis and perpendicular to the X axis. The steering angle 520 may then be adjusted to match the angle 512. In this way, an ultrasound beam emitted from an ultrasound probe may be adjusted to be perpendicular to an anatomical feature of interest (e.g., an angle of incidence may be adjusted to be at, or near, 90°) by adjusting a steering angle of the ultrasound beam based on feedback from a gyro sensor included in the ultrasound probe.

Referring now to FIG. 6A, an example user interface display 600 of a display device 602 is depicted. In one example, the display device 602 may be the display device 118 of the ultrasound imaging system 100 shown in FIG. 1. The example user interface display 600 may include an ultrasound image 604 depicting a tendon in a shoulder of a subject, the ultrasound image 604 generated from ultrasound imaging data received by the ultrasound probe (e.g., 106). As shown, the ultrasound image 604 may not clearly depict one or more image aspects of the tendon. An operator of the ultrasound imaging system may infer that the ultrasound image 604 provides a suboptimal depiction of the tendon via a color bar 606. The color bar 606 may be set to a first color or a second color based on feedback from the gyro sensor (e.g., 105) included in the ultrasound probe. For example, when the steering angle of the ultrasound probe is outside of the steering angle range determined for optimal imaging (e.g., determined to adjust the angle of incidence to 90°), the color bar 606 may be set to the first color, as shown in FIG. 6A. A verbal message 608 may further indicate that the steering angle is not currently at an optimal value. The operator may then manually adjust the steering angle towards the steering angle range or the controller (e.g., 116) may automatically adjust the steering angle towards the steering angle range.

Referring now to FIG. 6B, an example user interface display 650 of display device 652 is depicted. In one example, the display device 652 may be the display device 118 of the ultrasound imaging system shown in FIG. 1. The example user interface display 650 may include an ultrasound image 654 depicting a tendon in a shoulder of a subject, the ultrasound image 654 generated from ultrasound imaging data received by the ultrasound probe (e.g., 106). As shown, the ultrasound image 654 more clearly depicts one or more image aspects of the tendon as compared to the one or more image aspects depicted by the ultrasound image 604 of FIG. 6A. An operator of the ultrasound imaging system may infer that the ultrasound image 654 provides a substantially optimal depiction of the tendon via a color bar 656. As with the color bar 606 of FIG. 6A, the color bar 656 may be set to the first color or the second color based on feedback from the gyro sensor (e.g., 105) included in the ultrasound probe. For example, when the steering angle of the ultrasound probe is within the steering angle range determined for optimal imaging (e.g., determined to adjust the angle of incidence to 90°), the color bar 656 may be set to the second color, as shown in FIG. 6B. In some examples, the ultrasound image 654 may result from the manual or automatic adjustment of the steering angle described with reference to FIG. 6A.

Referring now to FIG. 7, a schematic diagram 700 of an example neural network 702 used for object detection and identification in image inputs (e.g., detection and identification of tendon damage in ultrasound image slices) is depicted. The neural network 702 may be included in a controller of an imaging system (e.g., system controller 116 of system 100 of FIG. 1) and/or in a system in electronic communication with the controller of the imaging system (or receiving data from the controller of the imaging system). The neural network 702 may be a convolutional neural network 702 (e.g., AlexNet, VGGNet, ResNet, etc.). Convolutional neural networks are a class of biologically-inspired deep neural networks that are powerful in image processing tasks. In particular, convolutional neural networks are modeled after the visual system of the brain. Unlike a “traditional” neural network, convolutional neural networks consist of layers organized in three dimensions and neurons in one layer are connected to only a subset of neurons in the next layer (instead of connecting to all neurons, such as in densely connected layers).

As shown in FIG. 7, the convolutional neural network 702 may consist of layers of computational units that process visual information hierarchically in a feed-forward manner. The output of each layer may include a plurality of feature maps 704 which may be understood as differently filtered versions of an input image 710. For example, the convolutional neural network 702 may include a plurality of convolutional layers 706 and pooling layers 708. Though the convolutional layers 706 and pooling layers 708 are shown in an alternating pattern in FIG. 7, in some embodiments, there may be more or less convolutional layers and/or more or less pooling layers and the number of convolutional layers and pooling layers may not be equal and may not be in the alternating pattern. The input image 710 (e.g., a preprocessed ultrasound image) may be input into the convolutional neural network 702. The input image 710, and each image of the feature maps 704, may be represented as a matrix of pixel intensity values. The matrices of pixel intensity values may be understood as the data which may be used by the convolutional neural network 702. Though a single input image 710 is shown in FIG. 7, in some examples, a plurality of sequential input images may be input into the convolutional neural network 702.

Convolution may occur at each of the convolutional layers 706. Convolution may be performed in order to extract features from the input image 710 (or the feature maps 704 in higher layers further along in the processing hierarchy). Convolution preserves the spatial relationship between pixels by mapping image features from a portion of a first layer to a portion of a second layer, using learning filters including a plurality of weights. Each convolutional layer 706 may include a collection of image filters, each of which extracts a certain feature from the given input image (e.g., 704, 710). Further, in some examples, each convolutional layer 706 may include rectified linear units for non-linear transformation and rectification following convolution. The output of each convolutional layer 706 may include a plurality of feature maps 704, each being a differently filtered version of the input image. In some examples, there may be one resulting feature map 704 per applied filter.

Pooling (e.g., spatial pooling, which may be 2×2 max pooling, or 2×2 sub sampling, in one example) may occur at each of the pooling layers 708. Pooling may be performed in order to reduce a dimensionality (e.g., size) of each feature map 704 while retaining or increasing certainty of feature identification. By pooling, a number of parameters and computations in the neural network 702 may be reduced, thereby controlling for overfitting, and a certainty of feature identification may be increased.

As shown in FIG. 7, following the first convolution, three feature maps 704 may be produced (however, it should be noted that this number may be representative and there may be greater than three feature maps in the first convolutional layer 706). Following the first pooling operation, the size of each feature map 704 may be reduced, though the number of feature maps 704 may be preserved. Then, during the second convolution, a larger number of filters may be applied and the output may be a correspondingly greater number of feature maps 704 in the second convolutional layer 706. Later layers along the processing hierarchy, shown by directional arrow 712, may be referred to as “higher” layers. The first few layers of the processing hierarchy may detect larger features while the later (higher) layers may pick up finer details and organize such details into more complex features. In some embodiments, a final output layer 714 may be fully connected (e.g., all neurons in the final output layer 714 may be connected to all neurons in the previous layer). The final output layer 714 may be used for classification following feature extraction via convolution and pooling. Thus, in some examples, the final output layer 714 may correspond to a classification module and earlier layers 706, 708 may correspond to a feature extraction module.

By training the convolutional neural network 702 on object recognition, the convolutional neural network 702 may develop a representation of the input image 710 which makes object information increasingly explicit along the processing hierarchy (as shown by arrow 712). Thus, along the processing hierarchy of the convolutional neural network 702, the input image 710 may be transformed into representations which increasingly emphasize the actual content of the input image 710 compared to its detailed pixel intensity values. Images reconstructed from the feature maps 704 of the higher layers in the convolutional neural network 702 may capture the high-level content in terms of objects and their arrangement in the input image 710 but may not constrain exact pixel intensity values of the content reconstructions. In contrast, image reconstructions from the lower layers may reproduce the exact pixel intensity values of the original input image 710. Thus, feature responses in the higher (e.g., deeper) layers of the convolutional neural network 702 may be referred to as the content representation.

In an exemplary embodiment, the convolutional neural network 702 may be trained to identify one or more image aspects of an anatomical feature depicted by an input image 710 (e.g., an ultrasound image) and thereby determine a most similar sample image from a plurality of sample images. Further, based on the most similar sample image, a degree of damage of the anatomical feature may be determined, for example, by obtaining a degree of damage of a corresponding anatomical feature depicted by the most similar sample image and inferring the degree of damage of the anatomical feature depicted by the imputed ultrasound image therefrom. Upon identification of the one or more image aspects and determination of the degree of damage, visual indicators for the one or more image aspects and/or the degree of damage may be generated, such that an operator of the ultrasound imaging system (e.g., 100) may be assisted in diagnosing the anatomical feature. In this way, the convolutional neural network may be trained to determine a degree of damage of an anatomical feature depicted by an ultrasound image, which may result in more consistent, predictable, and accurate diagnoses.

Referring now to FIG. 8, a diagram 800 depicts exemplary ultrasound images 810, 812, 814, 816, and 818 respectively depicting tendons having varying degrees of damage (e.g., such as those which may be displayed at the display device 118 of the ultrasound imaging system 100 shown in FIG. 1). Specifically, the ultrasound image 810 depicts a tendon having 0% damage (e.g., no rupture), the ultrasound images 812, 814, and 816 respectively depict tendons having 20% damage, 50% damage, and 80% damage (e.g., partial rupture), and the ultrasound image 818 depicts a tendon having 100% damage (e.g., complete rupture). Further, each of the ultrasound images depicting a damaged tendon (e.g., 812, 814, 816, 818) may be provided with a respective visual indication of the degree of damage superimposed thereon. As shown, graphical bars 822, 824, 826, 828 visually depict the degree of damage, where each graphical bar 822, 824, 826, 828 may be proportionally filled according to the degree of damage. Specifically, the graphical bar 822 indicates 20% damage, the graphical bar 824 indicates 50% damage, the graphical bar 826 indicates 80% damage, and the graphical bar 826 indicates 100% damage. In some examples, though not shown at FIG. 8, the ultrasound image 810 may further be provided with an empty graphical bar indicating 0% damage. In this way, an operator of the ultrasound imaging system may be provided with a degree of damage of a tendon in an intuitive visual format, which may supplement or supplant a numerical value or verbal message.

Referring now to FIG. 9, an example user interface display 900 of a display device 902 is depicted. In one example, the display device 902 may be the display device 118 of the ultrasound imaging system 100 shown in FIG. 1. The example user interface display 900 may include an ultrasound image 908 depicting a tendon in a shoulder of a subject, the ultrasound image 908 generated from ultrasound imaging data received by the ultrasound probe (e.g., 106). Visual indicators, such as a graphical bar 910 of a degree of damage of the tendon (e.g., the graphical bars 822, 824, 826, 828 of FIG. 8) and/or arrows 912 indicating image aspects identified by a trained neural network (e.g., the convolutional neural network 702 of FIG. 7), may be superimposed on the ultrasound image 908 to assist an operator of the ultrasound imaging system in diagnosing the tendon. Additionally, a color bar 914 may be provided to indicate to the operator whether a steering angle of the ultrasound probe (e.g., 106) is adjusted to a steering angle range determined to provide optimal imaging (e.g., adjusted to provide an angle of incidence at, or near, 90°), a message box 916 may provide verbal and numerical information to the operator (e.g., the steering angle, the degree of damage of the tendon, etc.), and a diagnosis recommendation 918 may be determined based on the degree of damage of the tendon and provided to the operator.

The ultrasound image 908 may further be matched, via the trained neural network, to a most similar sample image 904, where the most similar sample image 904 may be aligned to the ultrasound image 908 for comparison (as shown). In the example user interface display 900 shown at FIG. 9, a drop-down menu 906 includes the most similar sample image 904, in addition to the next most similar sample images, to the ultrasound image 908. As shown, the most similar sample image 904 is selected for expansion and alignment with the ultrasound image 908. In some examples, the next most similar sample images may be other sample images determined to match the ultrasound image 908 within a matching threshold. In other examples, the next most similar sample images may be a fixed or user-requested number of the next most similar sample images (e.g., the next two most similar sample images, as shown in FIG. 9). In this way, an operator of an ultrasound probe may be enabled to optimally generate an ultrasound image of an anatomical feature (e.g., by utilizing a gyro sensor to assist adjustment of a steering angle the ultrasound probe) and may be provided, via a trained neural network, with a most similar sample image to the generated image from which a degree of damage of the anatomical feature and a diagnosis recommendation may be determined.

In this way, a tendon rupture may be identified and diagnosed via a trained neural network implemented in an ultrasound imaging system. In one example, an image may be generated from acquired ultrasound imaging data and paired with a sample image depicting a similarly damaged tendon. Each of the generated image and the sample image may then be simultaneously displayed to a user of the ultrasound imaging system for comparison. A technical effect of pairing the sample image to the generated image is that a degree of damage of the tendon may be determined based on the sample image. A diagnosis recommendation may further be provided based on the determined degree of damage. In some examples, an ultrasound probe of the ultrasound imaging system may include a gyro sensor. A technical effect of incorporating the gyro sensor in this way is that an angle of incidence of the ultrasound probe may be determined in real-time, facilitating adjustment of a steering angle of the ultrasound probe for optimal imaging of the tendon. As such, embodiments of the ultrasound imaging system of the present disclosure may effect more consistent, predictable, and accurate treatment of damaged tendons.

In one embodiment, a method comprises acquiring an ultrasound image of an anatomical feature, pairing, via a trained neural network, the acquired ultrasound image to a sample image of a sample anatomical feature, determining a degree of damage of the anatomical feature based on the sample image, and displaying the acquired ultrasound image and the sample image simultaneously. In a first example of the method, the method further comprises identifying, via the trained neural network, one or more image aspects of the anatomical feature based on the sample image, and labelling the one or more identified image aspects of the anatomical feature on the acquired ultrasound image. In a second example of the method, optionally including the first example of the method, identifying the one or more image aspects of the anatomical feature based on the sample image comprises pairing a corresponding one of one or more predetermined image aspects of the sample anatomical feature to each of the one or more image aspects of the anatomical feature. In a third example of the method, optionally including one or more of the first and second examples of the method, the method further comprises displaying an indication of the degree of damage. In a fourth example of the method, optionally including one or more of the first through third examples of the method, the sample image is determined by the trained neural network to be a most similar sample image to the acquired ultrasound image out of a plurality of sample images. In a fifth example of the method, optionally including one or more of the first through fourth examples of the method, the trained neural network outputs a level of confidence in the most similar sample image, the level of confidence based on feedback from a gyro sensor indicating an angle of incidence at which the ultrasound image is acquired. In a sixth example of the method, optionally including one or more of the first through fifth examples of the method, each of the anatomical feature and the sample anatomical feature is a tendon in a shoulder of a subject.

In another embodiment, a method comprises training a neural network to determine a degree of damage of a tendon depicted by an ultrasound image, wherein determining the degree of damage comprises selecting, from a plurality of sample images, a most similar sample image to the ultrasound image, obtaining a degree of damage of a tendon depicted by the most similar sample image, and determining the degree of damage of the tendon depicted by the ultrasound image based on the degree of damage of the tendon depicted by the most similar sample image, receiving a particular ultrasound image depicting a particular tendon, and determining a degree of damage of the particular tendon depicted by the particular ultrasound image using the trained neural network. In a first example of the method, the trained neural network is a convolutional neural network. In a second example of the method, optionally including the first example of the method, the method further comprises displaying the particular ultrasound image while providing the most similar sample image for comparison. In a third example of the method, optionally including one or more of the first and second examples of the method, the method further comprises receiving the plurality of sample images, each of the plurality of sample images depicting a respective sample tendon, the sample tendon being associated with a sample degree of damage, wherein the neural network is trained based on the received plurality of sample images. In a fourth example of the method, optionally including one or more of the first through third examples of the method, the sample degrees of damage of the plurality of sample images range from no tendon damage to complete tendon rupture. In a fifth example of the method, optionally including one or more of the first through fourth examples of the method, each of the sample degrees of damage of the plurality of sample images is determined by one or more medical professionals.

In yet another embodiment, a medical imaging system comprises an ultrasound probe, a memory storing a plurality of sample image slices and a trained neural network configured to separate visual characteristics from content of an image, a display device, and a processor configured with instructions in non-transitory memory that when executed cause the processor to acquire imaging data from the ultrasound probe, generate, from the imaging data, an image slice depicting a tendon of a subject, and responsive to the trained neural network matching, within a matching threshold, one of the plurality of sample image slices to the generated image slice, determine a degree of damage of the tendon based on the matched sample image slice, simultaneously display, via the display device, the generated image slice aligned with the matched sample image slice, and provide, at the display device, a diagnosis of the tendon of the subject based on the degree of damage. In a first example of the medical imaging system, the ultrasound probe comprises a gyro sensor, and generating the image slice comprises obtaining a desired imaging plane, determining, via the gyro sensor, an orientation of the ultrasound probe, determining a steering angle range based on the orientation and the desired imaging plane, responsive to a steering angle of the ultrasound probe being outside of the steering angle range, generating the image slice with a first notification indicating the steering angle outside of the steering angle range, and responsive to the steering angle being within the steering angle range, generating the image slice with a second notification indicating the steering angle within the steering angle range. In a second example of the medical imaging system, optionally including the first example of the medical imaging system, the first notification comprises a color bar set to a first color, and the second notification comprises the color bar set to a second color. In a third example of the medical imaging system, optionally including one or more of the first and second examples of the medical imaging system, the processor is further configured to, responsive to none of the plurality of sample image slices matching the generated image slice within the matching threshold, displaying, via the display device, the generated image slice and a notification indicating no matching sample image slice was determined. In a fourth example of the medical imaging system, optionally including one or more of the first through third examples of the medical imaging system, the medical imaging system further comprises responsive to the trained neural network matching, within the matching threshold, a subset of the plurality of sample image slices to the generated image slice, determine, via the trained neural network, a most similar sample image slice to the generated image slice from the subset of the plurality of sample image slices, determine the degree of damage of the tendon based on the most similar sample image slice, simultaneously display, via the display device, the generated image slice aligned with the most similar sample image slice, provide, at the display device, remaining sample image slices in the subset of sample image slices in addition to the most similar sample image slice, and provide, at the display device, a diagnosis of the tendon of the subject based on the degree of damage. In a fifth example of the medical imaging system, optionally including one or more of the first through fourth examples of the medical imaging system, the processor is further configured to superimpose a visual indication of the degree of damage on one or both of the generated image slice and the matched sample image slice. In a sixth example of the medical imaging system, optionally including one or more of the first through fifth examples of the medical imaging system, providing the diagnosis of the tendon of the subject based on the degree of damage comprises, responsive to the degree of damage being greater than a diagnosis threshold, diagnosing the tendon as damaged, and recommending a surgical procedure to repair the tendon.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
acquiring an ultrasound image of an anatomical feature;
pairing, via a trained neural network, the acquired ultrasound image to a sample image of a sample anatomical feature;
determining a degree of damage of the anatomical feature based on the sample image; and
displaying the acquired ultrasound image and the sample image simultaneously.

2. The method of claim 1, further comprising:
identifying, via the trained neural network, one or more image aspects of the anatomical feature based on the sample image; and
labelling the one or more identified image aspects of the anatomical feature on the acquired ultrasound image.

3. The method of claim 2, wherein identifying the one or more image aspects of the anatomical feature based on the sample image comprises pairing a corresponding one of one or more predetermined image aspects of the sample anatomical feature to each of the one or more image aspects of the anatomical feature.

4. The method of claim 1, further comprising displaying an indication of the degree of damage.

5. The method of claim 1, wherein the sample image is determined by the trained neural network to be a most similar sample image to the acquired ultrasound image out of a plurality of sample images.

6. The method of claim 5, wherein the trained neural network outputs a level of confidence in the most similar sample image, the level of confidence based on feedback from a gyro sensor indicating an angle of incidence at which the ultrasound image is acquired.

7. The method of claim 1, wherein each of the anatomical feature and the sample anatomical feature is a tendon in a shoulder of a subject.

8. A method, comprising:
training a neural network to determine a degree of damage of a tendon depicted by an ultrasound image, wherein determining the degree of damage comprises:
selecting, from a plurality of sample images, a most similar sample image to the ultrasound image;
obtaining a degree of damage of a tendon depicted by the most similar sample image; and
determining the degree of damage of the tendon depicted by the ultrasound image based on the degree of damage of the tendon depicted by the most similar sample image;
receiving a particular ultrasound image depicting a particular tendon; and
determining a degree of damage of the particular tendon depicted by the particular ultrasound image using the trained neural network.

9. The method of claim 8, wherein the trained neural network is a convolutional neural network.

10. The method of claim 8, further comprising displaying the particular ultrasound image while providing the most similar sample image for comparison.

11. The method of claim 8, further comprising:
receiving the plurality of sample images, each of the plurality of sample images depicting a respective sample tendon, the sample tendon being associated with a sample degree of damage,
wherein the neural network is trained based on the received plurality of sample images.

12. The method of claim 11, wherein the sample degrees of damage of the plurality of sample images range from no tendon damage to complete tendon rupture.

13. The method of claim 11, wherein each of the sample degrees of damage of the plurality of sample images is determined by one or more medical professionals.

14. A medical imaging system, comprising:
an ultrasound probe;
a memory storing a plurality of sample image slices and a trained neural network configured to separate visual characteristics from content of an image;
a display device; and
a processor configured with instructions in non-transitory memory that when executed cause the processor to:
acquire imaging data from the ultrasound probe;
generate, from the imaging data, an image slice depicting a tendon of a subject; and
responsive to the trained neural network matching, within a matching threshold, one of the plurality of sample image slices to the generated image slice:
determine a degree of damage of the tendon based on the matched sample image slice,
simultaneously display, via the display device, the generated image slice aligned with the matched sample image slice, and
provide, at the display device, a diagnosis of the tendon of the subject based on the degree of damage.

15. The medical imaging system of claim 14, wherein
the ultrasound probe comprises a gyro sensor; and
generating the image slice comprises:
obtaining a desired imaging plane;
determining, via the gyro sensor, an orientation of the ultrasound probe;
determining a steering angle range based on the orientation and the desired imaging plane;
responsive to a steering angle of the ultrasound probe being outside of the steering angle range, generating the image slice with a first notification indicating the steering angle outside of the steering angle range; and
responsive to the steering angle being within the steering angle range, generating the image slice with a second notification indicating the steering angle within the steering angle range.

16. The medical imaging system of claim 15, wherein
the first notification comprises a color bar set to a first color, and
the second notification comprises the color bar set to a second color.

17. The medical imaging system of claim 14, wherein the processor is further configured to, responsive to none of the plurality of sample image slices matching the generated image slice within the matching threshold, displaying, via the display device, the generated image slice and a notification indicating no matching sample image slice was determined.

18. The medical imaging system of claim 14, further comprising:
responsive to the trained neural network matching, within the matching threshold, a subset of the plurality of sample image slices to the generated image slice:
determine, via the trained neural network, a most similar sample image slice to the generated image slice from the subset of the plurality of sample image slices,
determine the degree of damage of the tendon based on the most similar sample image slice,
simultaneously display, via the display device, the generated image slice aligned with the most similar sample image slice,
provide, at the display device, remaining sample image slices in the subset of sample image slices in addition to the most similar sample image slice, and
provide, at the display device, a diagnosis of the tendon of the subject based on the degree of damage.

19. The medical imaging system of claim 14, wherein the processor is further configured to superimpose a visual indication of the degree of damage on one or both of the generated image slice and the matched sample image slice.

20. The medical imaging system of claim 14, wherein providing the diagnosis of the tendon of the subject based on the degree of damage comprises, responsive to the degree of damage being greater than a diagnosis threshold:
diagnosing the tendon as damaged, and
recommending a surgical procedure to repair the tendon.
